SEQUENCE LISTING (2 sequences)

SEQ ID NO: 1 - DNA, 25 bp, Artificial sequence
  Forward primer
atgtctaataaaaaacagtcaaata

SEQ ID NO: 2 - DNA, 21 bp, Artificial sequence
  Reverse primer
cttcttagatgtaagctgttc

(19) United States
(12) Patent Application Publication
(10) Pub. No.: US 2013/0007911 A1
(43) Pub. Date: Jan. 3, 2013

(54) ENGINEERING MALE STERILITY OR NON-TRANSGENIC POLLEN

(75) Inventor: C. NEAL STEWART, JR., Knoxville, TN (US)

(73) Assignee: UNIVERSITY OF TENNESSEE RESEARCH FOUNDATION, Knoxville, TN (US)

(21) Appl. No.: 13/539,601

(22) Filed: Jul. 2, 2012

Related U.S. Application Data
(60) Provisional application No. 61/503,919, filed on Jul. 1, 2011.

(51) Int. Cl. C12N 15/82; A01H 5/10; A01H 5/00; C12N 5/10; C12N 1/00

(52) U.S. Cl. 800/278; 435/419; 435/320.1; 435/317.1; 800/298; 800/305; 800/307; 800/309; 800/310; 800/312; 800/313; 800/314; 800/315; 800/317; 800/317.1; 800/317.2; 800/317.3; 800/317.4; 800/318; 800/320; 800/320.1; 800/320.2; 800/320.3; 800/322

(57) ABSTRACT

The present invention relates to methods of blocking or reducing genetically modified plant (GMO) pollen flow using a “non-lethal” approach. In this aspect, at least one transgenic polynucleotide of interest is linked to a pollen-ablation construct as described herein. The pollen-ablation construct contains a polynucleotide encoding a restriction enzyme that renders the transgenic pollen unable to fertilize a sexually compatible ovule.

### CROSS-REFERENCE TO RELATED APPLICATION

This application claims the benefit of U.S. Provisional Application Ser. No. 61/503,919, filed Jul. 1, 2011, the disclosure of which is hereby incorporated by reference in its entirety, including all figures, tables and amino acid or nucleic acid sequences.

This invention was made with government support under DE-AC05-00OR22725 awarded by BioEnergy Science Center. The government has certain rights in the invention.

### BACKGROUND OF THE INVENTION

Developing containment strategies has been considered as a crucial step for genetically engineered (GE) crop cultivation. Most physical containment strategies including building fences, setting isolation distances, and manually removing flowers appeared to have very limited use (Moon et al., 2010). Biological strategies have been considered as more efficient and reliable methods to contain unwanted transgene escape from GE crops. Male sterility has been most extensively studied among many other biocontainment strategies and commercially utilized. Male sterility was initially used in plant breeding for the production of F₁ hybrids. In the reproductive cycles of higher plants, viable pollen is required for successful pollen germination, tube growth, and eventual double-fertilization via transmission of the sperm cells to the ovule. Transgene escape and introgression through pollen could be completely prevented if pollen were rendered nonviable. Multiple methods have been used to decrease pollen fertility via genic or cytoplasmic male sterility. Disrupting pollen development through genetic engineering has been suggested for suppressing transgene escape and introgression (Daniell, 2002; Feil et al., 2003). For example, many male sterile plants have been genetically engineered using constructs that disrupt the tapetum, a layer of cells found within the pollen sac, essential for pollen development (reviewed in Daniell, 2002). The first transgenic male sterile plant was generated by genetic engineering of tobacco plants with the chimeric ribonuclease gene (Mariani et al., 1990). Most genic male sterile plants have been achieved by using tapetum-specific promoters to drive the expression of toxic bacterial genes (e.g. Barnase from Bacillus amyloliquefaciens, diphtheria toxin A), resulting in no pollen formation (Hird et al., 1993; Koltunow et al., 1990; Lee et al., 2003). Since then, several genetic engineering efforts have been aimed at developing genic male sterility in plants. These include using cytotoxic barnase gene expression in pollen or anthers of poplar (Populus) trees and Kalanchoe blossfeldiana (Wei et al., 2007; García-Sogo et al., 2010).

Since genic male sterility strategies inhibit development of anther or pollen, the lack of pollen could create negative ecological impacts on pollen-feeding insects (Mlynárová et al., 2006). Another mean to generate male sterile plants is cytoplasmic male sterility (CMS) (Chase, 2006). CMS that blocks the production of functional pollen was resulted from mutations in plant mitochondrial genome (Hanson & Bentolila, 2004). CMS plants are thought to have utility for limiting transgene flow via pollen dispersal (Feil et al., 2003). More recently, genetically engineered CMS has been developed for transgene biocontainment (Ruiz & Daniell, 2005). This started with the successful genetic engineering of the tobacco (Nicotiana tabacum L.) chloroplast genome with the phaA gene coding for β-ketothiolase, which is known to confer cytoplasmic male sterility (Ruiz & Daniell, 2005). A potential drawback of using CMS as a biocontainment tool is the potential for transmission of the transgene from the cytoplasm to the nucleus. Transmission of paternal plastids and mitochondria in crosses involving parents with an alien cytoplasm occurs at low frequency (10⁻⁴ to 10⁻⁵), and even less frequent transmission is expected under field conditions (Svab & Maliga, 2007). Also the loss of fertility in a CMS breeding plant population could eventually be restored under natural conditions (Schnable & Wise, 1996). Plastid transformation is a method of male sterility using maternal inheritance feature. Maternally inherited plant plastid genome in most crop species provides several advantages in genetic engineering such as high level of transgene expression and express of multiple operons in the genome (Maliga, 2004). Since plastids are not maternally inherited in some plant species, the use of plastid-based male sterility may be limited to certain plant species (Hagemann, 2004). Despite its potential for biocontainment, plastid transformation has only been successfully established in limited numbers of plant species.

Pollen ablation has been demonstrated by expression of the diphtheria toxin gene under the control of the LAT52 pollen-specific and putative pectin esterase promoter in tobacco (Twell, 1995; Uk et al., 1998). Transgenic events containing single copy of the diphtheria toxin A-chain (DTx-A) gene have shown 50% aborted pollen and 50% normal pollen as expected (Uk et al., 1998). However, there could be concerns about the expression of such toxin genes that might negatively affect to the pollinators or even human consumers.

Restriction endonucleases are typically classified into three classes (I, II, and III) based on their enzymology and cofactor requirements (Wilson, 1988). Type II restriction endonucleases are the best understood, but unique among the classes in that they consist of separate endonuclease and methylase enzymes. For decades, molecular biologists have relied on the utility of type II restriction endonucleases for routine DNA manipulation in the laboratory. Among the type II systems, the EcoRI restriction endonuclease is one of the most studied and well characterized. This restriction enzyme recognizes the nucleotide sequence 5′-GAATTC-3′, requires Mg²⁺, functions as a homodimer, and creates a double-strand break at the site (Wilson, 1988). Barnes and Rine (1985) demonstrated nuclear entry and the resultant cell death associated with EcoRI expression in yeast, Saccharomyces cerevisiae. Induced expression of the EcoRI restriction endonuclease was lethal to transformed Escherichia coli containing a plasmid carrying the EcoRI gene and suppressed the growth of the cells (Gholizadeh et al., 2010). The expression of any type II restriction endonucleases in plants has not been reported. Here, we report first overexpression of the EcoRI restriction endonuclease in plants, particularly in pollen, resulting in pollen ablation and/or infertility. This pollen ablation and/or infertility by overexpression of the EcoRI could be used as a biocontainment strategy to prevent pollen-mediated transgene escape and introgression.

### BRIEF SUMMARY OF THE INVENTION

Methods and constructs for pollen ablation and/or infertility in transgenic plants are provided. In various embodiments, a promoter directing expression to pollen or anther is operably linked to the expression of a restriction enzyme, the expression of which renders the transgenic pollen grains unable to fertilize a sexually compatible ovule.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Pollen-specific ablation T-DNA engineered into tobacco.

FIG. 2. Model for selective male sterility in transgenic plants. This illustrates the difference between male sterile plants and selective male sterile plants with ablated pollen compared to a hemizygous transgenic plant.

FIG. 3. Microscopic images of pollen from transgenic EcoRI events. Transgenic EcoRI events were transformed with a vector containing the LAT52 pollen-specific promoter, the EcoRI gene, and GFP gene as a visual marker. All images were taken under a FITC filtered epifluorescence microscopy with white and blue light excitation at 200× magnification. Exposure time was 16.7 ms and 3 s for white light and blue light, respectively.

FIG. 4. Percentage of GFP positive pollen in transgenic EcoRI events. GFP positive pollen grains were detected and counted via the flow cytometry (FCM)-based transgenic pollen screening method. Percentage of GFP positive pollen ranged from 0 to 0.2 among nine different transgenic events, while the GFP control had 64% and non-transgenic Xanthi had no GFP positives. GFP control pollen samples were collected from a mixture of hemizygous and homozygous plants. GFP positive pollen percentage was acquired based on 3,000 pollen counting for each measurement with 3 replications. Error bars represent standard deviation of the means.

FIGS. 5A-5E. Phenotypes of a representative T1 transgenic event expressing EcoRI in pollen event 4 (EcoRI), and a nontransgenic tobacco (Xanthi). FIG. 5A: Plants three months post-germination; FIGS. 5B and 5C: Flower buds; FIG. 5D: Lateral view of flowers; FIG. 5E: Front view of flowers.

FIG. 6. Relative expression of EcoRI gene in mature pollen and young leaves. Mature pollen was collected within 2 hours of anthesis and opened but unexpanded young leaves were collected for RNA extraction. Total RNA was extracted from plant tissues using TriReagent. QPCR (Quantitative Real-Time Polymerase Chain Reaction) reactions were performed using 2× Power SYBRR Green QPCR Master Mix. Data were analyzed using the SDS 2.3 software (Applied Biosystems) and relative transcript expression levels of each target were normalized with respect to the tobacco Ubiquitin4 gene (Accession no. X77456). Results were analyzed using standard curve method.

### DETAILED DESCRIPTION OF THE INVENTION

One aspect of the present invention relates to methods of blocking or reducing genetically modified plant (GMO) pollen flow using a “non-lethal” approach. In this aspect, at least one transgenic polynucleotide of interest is linked to a pollen-ablation construct as described herein. The pollen-ablation construct contains a polynucleotide encoding a restriction enzyme that renders the transgenic pollen unable to fertilize a sexually compatible ovule.

Plants suitable for introduction of the pollen-ablation constructs disclosed herein can be monocots or dicots. Non-limiting examples of suitable plants include, but are not limited to, maize, canola, tobacco, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, switchgrass, rapeseed, clover, tobacco, turfgrass, carrot, cotton, alfalfa, rice, potato, eggplant, cucumber, Arabidopsis thaliana, and woody plants such as coniferous and deciduous trees. Thus, a transgenic plant or genetically modified plant cell of the invention can be an angiosperm or gymnosperm. The methods of the invention are particularly useful when applied to use in crops of corn, sorghum, rice, and other grasses.

A “heterologous polynucleotide” or a “heterologous nucleic acid” refers to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, these terms refer to a DNA segment which is foreign or heterologous to the cell, or homologous to the cell but in a position within the host cell nucleic acid in which the element is not ordinarily found.

As used herein, the terms “nucleic acid” or “polynucleotide” refer to deoxyribonucleotides or ribonucleotides and polymers thereof in either single- or double-stranded form. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

A “restriction enzyme”, as used herein, refers to any enzyme capable of cleaving nucleic acids, such as DNA or RNA. In certain embodiments, the restriction enzyme is a type I, type II or type III restriction endonuclease. Various other embodiments provide for the use of one or more restriction enzyme selected from: AarI; BanII; BseGI; BspPI; CfrI; EcoNI; Hsp92II; NlaIV; RsaI; TaiI; AasI; BbsI; BseJI; BspTI; ClaI; EcoO109I; I-PpoI; NmuCI; RsrII; TaqaI; AatII; BbuI; BseLI; BsrBI; CpoI; EcoRI; KasI; NotI; SacI; TaqI; Acc65I; BbvCI; BseMI; BsrDI; Csp45I; EcoRV; Kpn2I; NruI; SacII; TasI; AccB7I; BbvI; BseMII; BsrFI; Csp6I; EheI; KpnI; NsbI; SalI; TatI; AccI; BceAI; BseNI; BsrGI; CspI; Esp3I; KspAI; NsiI; SapI; TauI; AccIII; BcgI; BseRI; BsrI; DdeI; FauI; LweI; NspI; SatI; TfiI; AciI; BciVI; BseSI; BsrSI; DpnI; Fnu4HI; MbiI; OliI; Sau3AI; TliI; AclI; BclI; BseXI; BssHII; DpnII; FokI; MboI; PacI; Sau96I; TrulI; AdeI; BcnI; BseYI; BssKI; DraI; FseI; MboII; PaeI; SbfI; Tru9I; AfeI; BcuI; BsgI; BssSI; DraIII; FspAI; MfeI; PaeR7I; ScaI; TseI; AflII; BfaI; Bsh1236I; Bst1107I; DrdI; FspI; MlsI; PagI; SchI; Tsp45I; AflIII; BfiI; Bsh1285I; Bst98I; EaeI; GsuI; MluI; Paul; ScrFI; Tsp509I; AgeI; BfmI; BshNI; BstAPI; EagI; HaeII; MlyI; PciI; SdaI; TspRI; AhdI; BfrBI; BshTI; BstBI; Eam1104I; HaeIII; MmeI; PdiI; SduI; Tth1111; AleI; BfuAI; BsiEI; BstEII; Eam1105I; HgaI; MnlI; PdmI; SexAI; TurboNaeI; Alol; BfuCI; BsiHKAI; BstF5I; EarI; HhaI; Mph1103I; Pfl23II; SfaNI; TurboNarI; AluI; BfuI; BsiWI; BstNI; EciI; HinlI; MscI; PflFI; SfcI; Van91I; Alw21I; BglI; BslI; BstOI; Ecl136II; Hin4I; MseI; PflMI; SfiI; VspI; Alw26I; BglII; BsmAI; BstUI; EclHKI; Hin6I; MslI; PfoI; SfoI; XagI; Alw44I; BlpI; BsmBI; BstXI; Eco105I; HincII; MspAlI; Plel; Sgfl; XapI; AlwI; Bme1390I; BsmFI; BstYI; Eco130I; HindIII; MspI; PmeI; SgrAI; XbaI; AlwNI; BoxI; BsmI; BstZI; Eco147I; Hinfl; MssI; PmlI; SinI; XceI; ApaI; BpiI; BsoBI; Bsu15I; Eco24I; HinPlI; MunI; PpiI; SmaI; XcmI; ApaLI; BplI; Bsp119I; Bsu36I; Eco31I; HpaI; Mva1269I; PpuMI; SmiI; XhoI; ApoI; Bpu10I; Bsp120I; BsuRI; Eco32I; HpaII; MvaI; PshAI; SmlI; XhoII; AscI; Bpu1102I; Bsp1286I; BtgI; Eco47I; HphI; MwoI; PsiI; SmuI; XmaI; AseI; BsaAI; Bsp1407I; BtsI; Eco47III; Hpy188I; NaeI; Psp1406I; SnaBI; XmaJI; AsiSI; BsaBI; Bsp143I; BveI; Eco52I; Hpy188III; NarI; Psp5II; SpeI; XmiI; AvaI; BsaHI; Bsp143II; Cac8I; Eco57I; Hpy8I; NciI; PspGI; SphI; XmnI; AvaII; BsaI; Bsp68I; CaiI; Eco57MI; Hpy99I; NcoI; PspOMI; SspI; AvrII; BsaJI; BspDI; CfoI; Eco72I; HpyCH4III; NdeI; PstI; StuI; BaeI; BsaMI; BspEI; Cfr10I; Eco81I; HpyCH4IV; NdeII; PsuI; StyD4I; BalI; BsaWI; BspHI; Cfr13I; Eco88I; HpyCH4V; NgoMIV; PsyI; StyI; BamHI; BsaXI; BspLI; Cfr42I; Eco91I; HpyF10VI; NheI; PvuI; SwaI; BanI; BseDI; BspMI; Cfr9I; EcoICRI; Hsp92I; N1aIII; PvuII; or TaaI.

Other embodiments provide for the use of one or more restriction enzymes corresponding to a subset of the list provided above where the restriction enzymes are selected from: AluI; ClaI; Eco47III; HaeIII; KpnI; NdeI; PstI; SacII; SfiI; XhoI; BamHI; DpnI; EcoRI; HindIII; MspI; NheI; RsaI; SalI; SmaI; XmaI; BglII; DpnII; EcoRV; HpaII; NcoI; NotI; SacI; Sau3AI; or XbaI.

Yet other embodiments provide for the use of a restriction enzyme such as EcoRI, HindIII, etc. As would be apparent, any restriction enzyme can be used in the practice of the invention and a listing of such restriction enzymes can be found, for example, at “The RESTRICTION ENZYME DATABASE” (REBASE™; world wide web site: rebase.neb.com/rebase/rebase.html).

A nucleotide segment is referred to as “operably linked” when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Generally, DNA sequences that are operably linked are contiguous, and in the case of a signal sequence both contiguous and in reading phase. However, enhancers need not be contiguous with the coding sequences whose transcription they control. Linking is accomplished by ligation at convenient restriction sites or at adapters or linkers inserted in lieu thereof. The expression cassette can include one or more enhancers in addition to the promoter. By “enhancer” is intended a cis-acting sequence that increases the utilization of a promoter. Such enhancers can be native to a gene or from a heterologous gene. Further, it is recognized that some promoters can contain one or more native, enhancers or enhancer-like elements. An example of one such enhancer is the 35S enhancer, which can be a single enhancer, or duplicated. See for example, McPherson et al, U.S. Pat. No. 5,322,938, which is hereby incorporated by reference in its entirety.

The term “pollen-specific promoter” refers to a nucleic acid sequence that regulates the expression of nucleic acid sequences selectively in the cells or tissues of a plant essential to pollen formation and/or function and/or limits the expression of a nucleic acid sequence to the period of pollen formation in the plant. It may express at higher levels in the pollen tissue compared to other plant tissue, may express highly in the pollen, express more in the pollen tissue than in other plant tissue, or express exclusively in the pollen tissue. Pollen-specific promoters include those promoters active during pollen development, as well as those promoters active during pollen germination or active in anther and/or pollen or in tissues that give rise to anther cells and/or pollen or pollen compartments, including but not limited to the amyloplastid, mitochondria, protein bodies, oil bodies or other compartments in pollen, including those that store energy sources and enzymes. Suitable pollen-specific promoters drive expression specifically, preferentially in pollen and may be expressed in other parts of the plant as well.

Pollen specific promoters include, for example, an MS45 gene promoter (U.S. Pat. No. 6,037,523), a 5126 gene promoter (U.S. Pat. No. 5,837,851), a BS7 gene promoter (WO 02/063021), an SB200 gene promoter, (WO 02/26789) a TA29 gene promoter (Nature 347:737 (1990)), a PG47 gene promoter (U.S. Pat. No. 5,412,085; U.S. Pat. No. 5,545,546; Plant J 3(2):261-271 (1993)), P67 or P95 (See US publication 20050246796) promoters, an SGB6 gene promoter (U.S. Pat. No. 5,470,359) a G9 gene promoter (U.S. Pat. Nos. 5,837,850; 5,589,610), or the like. Additional tissue-specific or stage-specific regulatory elements include the Zn13 promoter, which is a pollen-specific promoter (Hamilton et al., Plant Mol. Biol. 18:211-218, 1992); and sperm cell-specific promoters. Pollen-specific promoters have also been identified in many other plant species such as maize, rice, tomato, tobacco, Arabidopsis, Brassica, and others (Odell, T. O., et al. (1985) Nature 313:810-812; Marrs, K. A., et al, (1993) Dev Genet, Vol. 14/1:27-41; Kim, (1992) Transgenic Res, Vol. 1/4:188-94; Carpenter, J. L., et al. (1992) Plant Cell Vol. 4/5:557-71; Albani, D. et al., (1992) Plant J. 2/3:331-42; Rommens, C. M., et al. (1992), Mol. Gen. Genet., Vol. 231/3:433-41; Kloeckener-Gruissem, et al., (1992) Embo J, Vol. 11/1:157-66; Hamilton, D. A. et al., (1992), Plant Mol Biol, Vol. 18/2:211-18; Kyozuka, J., et al. (1991), Mol. Gen. Genet., Vol. 228/1-2:40-8; Albani, D. et. al (1991) Plant Mol Biol Vol. 16/4:501-13; Twell, D. et al. (1991) Genes Dev. 5/3:496-507; Thorsness, M. K. et al., (1991) Dev. Biol Vol. 143/1:173-84; McCormick, S. et al. (1991) Symp Soc Exp Biol Vol. 45:229-44; Guerrero, F. D. et al. (1990) Mol Gen Genet Vol 224/2:161-8; Twell, D. et al., (1990) Development Vol. 109/3:705-13; Bichler, J. et al. (1990), Eur J Biochem Vol. 190/2:415-26; van Tunen, et al. (1990), Plant Cell Vol 2/5:393-401; Siebertz, B. et al., (1989) Plant Cell Vol 1/10:961-8; Sullivan, T. D. et al, (1989) Dev Genet Vol 10/6:412-24; Chen, J. et al. (1987), Genetics Vol 116/3:469-77). Several other examples of pollen-specific promoters can be found in GenBank. Additional promoters are also provided in U.S. Pat. Nos. 5,086,169; 5,756,324; 5,633,438; 5,412,085; 5,545,546; and 6,172,279. Each of the patents and references cited in this paragraph is expressly incorporated by reference in its entirety, particularly with respect to the pollen-specific promoters disclosed within each respective document.

The term “recombinant” is used herein to refer to a nucleic acid molecule that is manipulated outside of a cell, including two or more linked heterologous nucleotide sequences.

The term “plant” is used broadly herein to include any plant at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

As used herein, the term “vector” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

The term “marker gene” or “marker” refers to a gene encoding a product that, when expressed, confers a phenotype or genotype on a transformed cell providing identification of cells expressing the marker.

In one aspect, the pollen-ablation construct including the pollen-specific promoter operably linked to a restriction enzyme may be linked to one or more transgenic polynucleotides of interest. Transgenic polynucleotides of interest include but are not limited to those which impact plant insecticide resistance, disease resistance, herbicide resistance, nutrition and cellulose content, abiotic stress resistance, for example, nitrogen fixation, yield enhancement genes, drought tolerance genes, cold tolerance genes, antibiotic resistance, genes complementing recessive agronomic traits such as recessive male sterility, and/or other marker genes.

Transgenic polynucleotides that confer resistance to insects or disease include but are not limited to the following: Bacillus thuringiensis toxin, a derivative thereof or a synthetic polypeptide modeled thereon. See, for example, Geiser et al., Gene 48: 109 (1986), who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Nos. 40098, 67136, 31995 and 31998. Other examples of Bacillus thuringiensis transgenes being genetically engineered are given in the following patents and patent applications and hereby are incorporated by reference: U.S. Pat. Nos. 5,188,960; 5,689,052; 5,880,275; WO 91/14778; WO 99/31248; WO 01/12731; WO 99/24581; and WO 97/40162; an insect-specific hormone or pheromone such as an ecdysteroid and juvenile hormone, a variant thereof, a mimetic based thereon, or an antagonist or agonist thereof. See, for example, the disclosure by Hammock et al., Nature 344: 458 (1990), of baculovirus expression of cloned juvenile hormone esterase, an inactivator of juvenile hormone; an insect-specific peptide which, upon expression, disrupts the physiology of the affected pest. For example, see the disclosures of Regan, J. Biol. Chem. 269: 9 (1994) (expression cloning yields DNA coding for insect diuretic hormone receptor); Pratt et al., Biochem. Biophys. Res. Comm. 163: 1243 (1989) (an allostatin is identified in Diploptera puntata); Chattopadhyay et al. (2004) Critical Reviews in Microbiology 30 (1): 33-54 2004; Zjawiony (2004) J Nat Prod 67 (2): 300-310; Carlini & Grossi-de-Sa (2002) Toxicon, 40 (11): 1515-1539; Ussuf et al. (2001) Curr Sci. 80 (7): 847-853; and Vasconcelos & Oliveira (2004) Toxicon 44 (4): 385-403. See also U.S. Pat. No. 5,266,317 to Tomalski et al., who disclose genes encoding insect-specific toxins; an enzyme responsible for a hyperaccumulation of a monoterpene, a sesquiterpene, a steroid, hydroxamic acid, a phenylpropanoid derivative or another non-protein molecule with insecticidal activity; an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23: 691 (1993), who teach the nucleotide sequence of a cDNA encoding tobacco hookworm chitinase, and Kawalleck et al., Plant Molec. Biol. 21: 673 (1993), who provide the nucleotide sequence of the parsley ubi4-2 polyubiquitin gene, and U.S. Pat. No. 6,563,020; a molecule that stimulates signal transduction. For example, see the disclosure by Botella et al., Plant Molec. Biol. 24: 757 (1994), of nucleotide sequences for mung bean calmodulin cDNA clones, and Griess et al., Plant Physiol. 104: 1467 (1994), who provide the nucleotide sequence of a maize calmodulin cDNA clone; hydrophobic moment peptide. See PCT application WO 95/16776 and U.S. Pat. No. 5,580,852 (disclosure of peptide derivatives of Tachyplesin which inhibit fungal plant pathogens) and PCT application WO 95/18855 and U.S. Pat. No. 5,607,914) (teaches synthetic antimicrobial peptides that confer disease resistance); a membrane permease, a channel former or a channel blocker. For example, see the disclosure by Jaynes et al., Plant Sci. 89: 43 (1993), of heterologous expression of a cecropin-beta lytic peptide analog to render transgenic tobacco plants resistant to Pseudomonas solanacearum; a viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28: 451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus; an insect-specific antibody or an immunotoxin derived therefrom. Thus, an antibody targeted to a critical metabolic function in the insect gut would inactivate an affected enzyme, killing the insect. Cf. Taylor et al., Abstract #497, SEVENTH INT′L SYMPOSIUM ON MOLECULAR PLANT-MICROBE INTERACTIONS (Edinburgh, Scotland, 1994) (enzymatic inactivation in transgenic tobacco via production of single-chain antibody fragments); a virus-specific antibody. See, for example, Tavladoraki et al., Nature 366: 469 (1993), who show that transgenic plants expressing recombinant antibody genes are protected from virus attack; a developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See Lamb et al., Bio/Technology 10: 1436 (1992). The cloning and characterization of a gene which encodes a bean endopolygalacturonase-inhibiting protein is described by Toubart et al., Plant J. 2: 367 (1992); a developmental-arrestive protein produced in nature by a plant. For example, Logemann et al., Bio/Technology 10: 305 (1992), have shown that transgenic plants expressing the barley ribosome-inactivating gene have an increased resistance to fungal disease; genes involved in the Systemic Acquired Resistance (SAR) Response and/or the pathogenesis related genes. Briggs, S., Current Biology, 5(2):128-131 (1995), Pieterse & Van Loon (2004) Curr. Opin. Plant Bio. 7(4):456-64 and Somssich (2003) Cell 113(7):815-6; Antifungal genes (Cornelissen and Melchers, P1. Physiol. 101:709-712, (1993) and Parijs et al., Planta 183:258-264, (1991) and Bushnell et al., Can. J. of Plant Path. 20(2): 137-149 (1998). Also see U.S. Pat. No. 6,875,907; Detoxification genes, such as for fumonisin, beauvericin, moniliformin and zearalenone and their structurally related derivatives. For example, see U.S. Pat. No. 5,792,931; Genes conferring resistance to nematodes. See WO 03/033651 and Urwin et. al., Planta 204:472-479 (1998), Williamson (1999) Curr Opin Plant Bio. 2(4):327-31; Genes such as rcg1 conferring resistance to Anthracnose stalk rot, which is caused by the fungus Colletotrichum graminiola. See M. Jung et al., Generation-means analysis and quantitative trait locus mapping of Anthracnose Stalk Rot genes in Maize, Theor. Appl. Genet. (1994) 89:413-418 which is incorporated by reference for this purpose, as well as U.S. Patent Application 60/675,664, which is also incorporated by reference.

Transgenic polynucleotides of interest that confer resistance to a herbicide, include without limitation, a herbicide that inhibits the growing point or meristem, such as an imidazolinone or a sulfonylurea. Exemplary genes in this category code for mutant ALS and AHAS enzyme as described, for example, by Lee et al., EMBO J. 7: 1241 (1988), and Miki et al., Theor. Appl. Genet. 80: 449 (1990), respectively. See also, U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference for this purpose; glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cycloshexones (ACCase inhibitor-encoding genes). See, for example, U.S. Pat. No. 4,940,835 to Shah et al., which discloses the nucleotide sequence of a form of EPSPS which can confer glyphosate resistance. U.S. Pat. No. 5,627,061 to Barry et al. also describes genes encoding EPSPS enzymes. See also U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; RE 36,449; RE 37,287 E; and 5,491,288; and international publications EP1173580; WO 01/66704; EP1173581 and EP1173582, which are incorporated herein by reference for this purpose. Glyphosate resistance is also imparted to plants that express a gene that encodes a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase. A DNA molecule encoding a mutant aroA gene can be obtained under ATCC accession No. 39256, and the nucleotide sequence of the mutant gene is disclosed in U.S. Pat. No. 4,769,061 to Comai. European Patent Application No. 0 333 033 to Kumada et al. and U.S. Pat. No. 4,975,374 to Goodman et al. disclose nucleotide sequences of glutamine synthetase genes which confer resistance to herbicides such as L-phosphinothricin. The nucleotide sequence of a phosphinothricin-acetyl-transferase gene is provided in European Patent No. 0 242 246 and 0 242 236 to Leemans et al. De Greef et al., Bio/Technology 7: 61 (1989), describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1; and 5,879,903, which are incorporated herein by reference for this purpose. Exemplary genes conferring resistance to phenoxy proprionic acids and cycloshexones, such as sethoxydim and haloxyfop, are the Acc1-S1, Acc1-S2 and Acc1-S3 genes described by Marshall et al., Theor. Appl. Genet. 83: 435 (1992). A herbicide that inhibits photosynthesis, such as a triazine (psbA and gs+ genes) and a benzonitrile (nitrilase gene) is also included. Przibilla et al., Plant Cell 3: 169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285: 173 (1992). Acetohydroxy acid synthase, which has been found to make plants that express this enzyme resistant to multiple types of herbicides, has been introduced into a variety of plants (see, e.g., Hattori et al. (1995) Mol Gen Genet. 246:419). Other genes that confer resistance to herbicides include: a gene encoding a chimeric protein of rat cytochrome P4507A1 and yeast NADPH-cytochrome P450 oxidoreductase (Shiota et al. (1994) Plant Physiol. 106(1):17-23), genes for glutathione reductase and superoxide dismutase (Aono et al. (1995) Plant Cell Physiol 36:1687, and genes for various phosphotransferases (Datta et al. (1992) Plant Mol Biol 20:619). Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; and 5,767,373; and international publication WO 01/12825.

Transgenic polynucleotides of interest also include those genes that confer or contribute to nutrition, cellulose content, or alter grain characteristic, such as an altered fatty acid, for example, by down-regulation of stearoyl-ACP desaturase to increase stearic acid content of the plant. See Knultzon et al., Proc. Natl. Acad. Sci. USA 89: 2624 (1992) and WO99/64579 (Genes for Desaturases to Alter Lipid Profiles in Corn), elevating oleic acid via FAD-2 gene modification and/or decreasing linolenic acid via FAD-3 gene modification (see U.S. Pat. Nos. 6,063,947; 6,323,392; 6,372,965 and WO 93/11245); altering conjugated linolenic or linoleic acid content, such as in WO 01/12800; altering LEC1, AGP, Dek1, Superal1, mi1ps, various 1pa genes such as 1pa1, Ipa3, hpt or hggt. For example, see WO 02/42424, WO 98/22604, WO 03/011015, U.S. Pat. No. 6,423,886, U.S. Pat. No. 6,197,561, U.S. Pat. No. 6,825,397, US2003/0079247, US2003/0204870, WO02/057439, WO03/011015 and Rivera-Madrid, R. et. al. Proc. Natl. Acad. Sci. 92:5620-5624 (1995). Transgenic polynucleotides of interest also include those that alter phosphorus content, for example, by the introduction of a phytase-encoding gene that would enhance breakdown of phytate, adding more free phosphate to the transformed plant. For example, see Van Hartingsveldt et al., Gene 127: 87 (1993), for a disclosure of the nucleotide sequence of an Aspergillus niger phytase gene; or those that up-regulate a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy et al., Maydica 35: 383 (1990) and/or by altering inositol kinase activity as in WO 02/059324, US2003/0009011, WO 03/027243, US2003/0079247, WO 99/05298, U.S. Pat. No. 6,197,561, U.S. Pat. No. 6,291,224, U.S. Pat. No. 6,391,348, WO2002/059324, US2003/0079247, WO98/45448, WO99/55882, WO01/04147. Other transgenic polynucleotides of interest include but are not limited to those that alter carbohydrates effected, for example, by altering a gene for an enzyme that affects the branching pattern of starch or a gene altering thioredoxin such as NTR and/or TRX (see U.S. Pat. No. 6,531,648 which is incorporated by reference for this purpose) and/or a gamma zein knock out or mutant such as cs27 or TUSC27 or en27 (See U.S. Pat. No. 6,858,778 and US2005/0160488, US2005/0204418; which are incorporated by reference for this purpose). See Shiroza et al., J. Bacteriol. 170: 810 (1988) (nucleotide sequence of Streptococcus mutans fructosyltransferase gene), Steinmetz et al., Mol. Gen. Genet. 200: 220 (1985) (nucleotide sequence of Bacillus subtilis levansucrase gene), Pen et al., Bio/Technology 10: 292 (1992) (production of transgenic plants that express Bacillus licheniformis alpha-amylase), Elliot et al., Plant Molec. Biol. 21: 515 (1993) (nucleotide sequences of tomato invertase genes), Sogaard et al., J. Biol. Chem. 268: 22480 (1993) (site-directed mutagenesis of barley alpha-amylase gene), and Fisher et al., Plant Physiol. 102: 1045 (1993) (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

Transgenic polynucleotides of interest also include those genes that confer or contribute to an altered grain characteristic include without limitation the those that alter antioxidant content or composition, such as alteration of tocopherol or tocotrienols. For example, see U.S. Pat. No. 6,787,683, US2004/0034886 and WO 00/68393 involving the manipulation of antioxidant levels through alteration of a phytl prenyl transferase (ppt), WO 03/082899 through alteration of a homogentisate geranyl geranyl transferase (hggt).

Also included are those that alter essential seed amino acids. For example, see U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US2003/0163838, US2003/0150014, US2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US2004/0025203 (UDPGdH), U.S. Pat. No. 6,194,638 (RGP).

Transgenic polynucleotides of interest also include but are not limited to genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. For example, see: WO 00/73475 where water use efficiency is altered through alteration of malate; U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, and WO9938977 describing genes, including CBF genes and transcription factors effective in mitigating the negative effects of freezing, high salinity, and drought on plants, as well as conferring other positive effects on plant phenotype; US2004/0148654 and WO01/36596 where abscisic acid is altered in plants resulting in improved plant phenotype such as increased yield and/or increased tolerance to abiotic stress; WO2000/006341 and WO04/090143 where cytokinin expression is modified resulting in plants with increased stress tolerance, such as drought tolerance, and/or increased yield. Also see WO0202776, WO2003052063, JP2002281975, U.S. Pat. No. 6,084,153, WO0164898, U.S. Pat. No. 6,177,275, and U.S. Pat. No. 6,107,547 (enhancement of nitrogen utilization and altered nitrogen responsiveness). For ethylene alteration, see US20040128719, US20030166197 and WO200032761. For plant transcription factors or transcriptional regulators of abiotic stress, see e.g. US20040098764 or US20040078852.

Transgenic polynucleotides of interests of interest also include but are not limited to marker genes or markers. A marker provides a means for screening a population of organisms or cells of an organism (e.g., plants or plant cells) to identify those having the marker and, therefore, the transgenic polynucleotide of interest. Also for with respect to non-food crops, a fluorescent protein may be preferred marker with which to facilitate selection. A selectable marker may confers a selective advantage to the cell, or to an organism (e.g., a plant) containing the cell, for example, the ability to grow in the presence of a negative selective agent such as an antibiotic or, for a plant, an herbicide. Examples of selectable markers include those that confer resistance to antimetabolites such as herbicides or antibiotics, for example, dihydrofolate reductase, which confers resistance to methotrexate (Reiss, Plant Physiol. (Life Sci. Adv.) 13:143-149, 1994; see also Herrera Estrella et al., Nature 303:209-213, 1983; Meijer et al., Plant Mol. Biol. 16:807-820, 1991); neomycin phosphotransferase, which confers resistance to the aminoglycosides neomycin, kanamycin and paromycin (Herrera-Estrella, EMBO J. 2:987-995, 1983) and hygro, which confers resistance to hygromycin (Marsh, Gene 32:481-485, 1984; see also Waldron et al., Plant Mol. Biol. 5:103-108, 1985; Zhijian et al., Plant Science 108:219-227, 1995); trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include, for example, a mutant EPSPV-synthase, which confers glyphosate resistance (Hinchee et al., BioTechnology 91:915-922, 1998), a mutant acetolactate synthase, which confers imidazolinone or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Examples of suitable selectable marker genes include, but are not limited to, genes encoding resistance to chloramphenicol (Herrera Estrella et al., EMBO J. 2:987-992, 1983); streptomycin (Jones et al., Mol. Gen. Genet. 210:86-91, 1987); spectinomycin (Bretagne-Sagnard et al., Transgenic Res. 5:131-137, 1996); bleomycin (Hille et al., Plant Mol. Biol. 7:171-176, 1990); sulfonamide (Guerineau et al., Plant Mol. Biol. 15:127-136, 1990); bromoxynil (Stalker et al., Science 242:419-423, 1988); glyphosate (Shaw et al., Science 233:478-481, 1986); phosphinothricin (DeBlock et al., EMBO J. 6:2513-2518, 1987), and the like. One option for use of a selective gene is a glufosinate-resistance encoding DNA and in one embodiment can be the phosphinothricin acetyl transferase (“PAT”), maize optimized PAT gene or bar gene under the control of the CaMV 35S or ubiquitin promoters. The genes confer resistance to bialaphos. See, Gordon-Kamm et al., Plant Cell 2:603; 1990; Uchimiya et al., BioTechnology 11:835, 1993; White et al., Nucl. Acids Res. 18:1062, 1990; Spencer et al., Theor. Appl. Genet. 79:625-631, 1990; and Anzai et al., Mol. Gen. Gen. 219:492, 1989). A version of the PAT gene is the maize optimized PAT gene, described at U.S. Pat. No. 6,096,947.

In addition, markers that facilitate identification of a plant cell containing the polynucleotide encoding the marker may be employed. Scorable or screenable markers are useful, where presence of the sequence produces a measurable product. Examples include a beta-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. The EMBO Journal vol. 6 No. 13 pp. 3901-3907); alkaline phosphatase. In a preferred embodiment, the marker used is beta-carotene or provitamen A (Ye et al, supra). The gene has been used to enhance the nutrition of rice, but in this instance it is employed instead as a screenable marker, and the presence of the gene linked to a gene of interest is detected by the golden color provided. Unlike the situation where the gene is used for its nutritional contribution to the plant, a smaller amount of the protein is needed. Other screenable markers include the anthocyanin/flavonoid genes in general (See discussion at Taylor and Briggs, The Plant Cell (1990)2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., Plant Cell (1996) 8: 1171-1179; Scheffler et al. Mol. Gen. Genet. (1994) 242:40-48) and maize C2 (Wienand et al., Mol. Gen. Genet. (1986) 203:202-207); the B gene (Chandler et al., Plant Cell (1989) 1:1175-1183), the pl gene (Grotewold et al, Proc. Natl. Acad. Sci. USA (1991) 88:4587-4591; Grotewold et al., Cell (1994) 76:543-553; Sidorenko et al., Plant Mol. Biol. (1999)39:11-19); the bronze locus genes (Ralston et al., Genetics (1988) 119:185-197; Nash et al., Plant Cell (1990) 2(11): 1039-1049), among others. Yet further examples of suitable markers include the cyan fluorescent protein (CYP) gene (Bolte et al. (2004) J. Cell Science 117: 943-54 and Kato et al. (2002) Plant Physiol 129: 913-42), the yellow fluorescent protein gene (PhiYFP™ from Evrogen; see Bolte et al. (2004) J. Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., Plant J. (1995) 8(5):777-84); and DsRed2 where plant cells transformed with the marker gene are red in color, and thus visually selectable (Dietrich et al. (2002) Biotechniques 2(2):286-293). Additional examples include a p-lactamase gene (Sutcliffe, Proc. Nat'l. Acad. Sci. U.S.A. (1978) 75:3737), which encodes an enzyme for which various chromogenic substrates are known (e.g., PADAC, a chromogenic cephalosporin); a xylE gene (Zukowsky et al., Proc. Nat'l. Acad. Sci. U.S.A. (1983) 80:1101), which encodes a catechol dioxygenase that can convert chromogenic catechols; an alpha-amylase gene (Ikuta et al., Biotech. (1990) 8:241); and a tyrosinase gene (Katz et al., J. Gen. Microbiol. (1983) 129:2703), which encodes an enzyme capable of oxidizing tyrosine to DOPA and dopaquinone, which in turn condenses to form the easily detectable compound melanin. Clearly, many such markers are available to one skilled in the art.

The promoter for driving expression of the transgenic polynucleotide of interest may be selected based on a number of criteria, including but not limited to what the desired use is for the transgenic polynucleotide of interest, what location in the plant is expression of the transgenic polynucleotide of interest desired, and at what level is expression of transgenic polynucleotide of interest desired or whether it needs to be controlled in another spatial or temporal manner. In one aspect, a promoter that directs expression to particular tissue may be desirable. When referring to a promoter that directs expression to a particular tissue is meant to include promoters referred to as tissue specific or tissue preferred. Included within the scope of the invention are promoters that express highly in the plant tissue, express more in the plant tissue than in other plant tissue, or express exclusively in the plant tissue. For example, “seed-specific” promoters may be employed to drive expression of a color marker. Specific-seed promoters include those promoters active during seed development, promoters active during seed germination, and/or that are expressed only in the seed. Seed-specific promoters, such as annexin, P34, beta-phaseolin, alpha subunit of beta-conglycinin, oleosin, zein, napin promoters have been identified in many plant species such as maize, wheat, rice and barley. See U.S. Pat. Nos. 7,157,629, 7,129,089, and 7,109,392. Such seed-preferred promoters further include, but are not limited to, Cim1 (cytokinin-induced message); cZ19B1 (maize 19 kDa zein); and milps (myo-inositol-1-phosphate synthase); (see WO 00/11177, herein incorporated by reference). The 27 kDa gamma-zein promoter is a preferred endosperm-specific promoter. The maize globulin-1 and oleosin promoters are preferred embryo-specific promoters. For dicots, seed-specific promoters include, but are not limited to, bean beta phaseolin, napin, beta-conglycinin, soybean lectin, cruciferin, and the like. For monocots, seed-specific promoters include, but are not limited to, promoters of the 15 kDa beta-zein, 22 kDa alpha-zein, 27 kDa gamma-zein, waxy, shrunken 1, shrunken 2, globulin 1, an LtpI, an Ltp2, and oleosin genes. See also WO 00/12733, where seed-preferred promoters from end1 and end2 genes are disclosed; herein incorporated by reference. Any suitable promoter can be used that directs expression of the transgene of interest, including, for example, a constitutively active promoter such as an ubiquitin promoter, which generally effects transcription in most or all plant cells.

The promoters useful in the present invention can include constitutive promoters, which generally are active in most or all tissues of a plant; inducible promoters, which generally are inactive or exhibit a low basal level of expression, and can be induced to a relatively high activity upon contact of cells with an appropriate inducing agent; tissue-specific (or tissue-preferred) promoters, which generally are expressed in only one or a few particular cell types (e.g., plant anther cells); and developmental- or stage-specific promoters, which are active only during a defined period during the growth or development of a plant. Often promoters can be modified, if necessary, to vary the expression level. Certain embodiments comprise promoters exogenous to the species being manipulated.

Exemplary constitutive promoters include the 35S cauliflower mosaic virus (CaMV) promoter (Odell et al. (1985) Nature 313:810-812), the maize ubiquitin promoter (Christensen et al. (1989) Plant Mol. Biol. 12:619-632 and Christensen et al. (1992) Plant Mol. Biol. 18:675-689); the core promoter of the Rsyn7 promoter and other constitutive promoters disclosed in WO 99/43838 and U.S. Pat. No. 6,072,050; rice actin (McElroy et al. (1990) Plant Cell 2:163-171); pEMU (Last et al. (1991) Theor. Appl. Genet. 81:581-588); MAS (Velten et al. (1984) EMBO J. 3:2723-2730); ALS promoter (U.S. Pat. No. 5,659,026); rice actin promoter (U.S. Pat. No. 5,641,876; WO 00/70067), maize histone promoter (Brignon et al., Plant Mol Bio 22(6):1007-1015 (1993); Rasco-Gaunt et al., Plant Cell Rep. 21(6):569-576 (2003)) and the like. Other constitutive promoters include, for example, those described in U.S. Pat. Nos. 5,608,144 and 6,177,611, and PCT publication WO 03/102198.

An inducible promoter/regulatory element is one that is capable of directly or indirectly activating transcription of one or more DNA sequences or genes in response to an inducer. The inducer can be a chemical agent such as a protein, metabolite, growth regulator, herbicide or phenolic compound; or a physiological stress, such as that imposed directly by heat, cold, salt, or toxic elements, or indirectly through the action of a pathogen or disease agent such as a virus; or other biological or physical agent or environmental condition. A plant cell containing an inducible promoter/regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression.

Any inducible promoter/regulatory element can be used in the instant invention (See Ward et al., Plant Mol. Biol. 22: 361-366, 1993). Non-limiting examples of such promoters/regulatory elements include: a metallothionein regulatory element, a copper-inducible regulatory element, or a tetracycline-inducible regulatory element, the transcription from which can be effected in response to divalent metal ions, copper or tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994). Inducible promoters/regulatory elements also include an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991; U.S. Pat. No. 6,504,082); a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; and the light-inducible promoter derived from the pea rbcS gene or pea psaDb gene (Yamamoto et al. (1997) Plant J. 12(2):255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90(20):9586-9590; Orozco et al. (1993) Plant Mol. Bio. 23(6): 1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible promoter/regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genet. 227:229-237, 1991). Stress inducible promoters include salt/water stress-inducible promoters such as P5CS (Zang et al. (1997) Plant Sciences 129:81-89); cold-inducible promoters, such as, corl5a (Hajela et al. (1990) Plant Physiol. 93:1246-1252), corl5b (Wlihelm et al. (1993) Plant Mol Biol 23:1073-1077), wscl20 (Ouellet et al. (1998) FEBS Lett. 423-324-328), ci7 (Kirch et al. (1997) Plant Mol. Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338), and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331-340). Certain promoters are inducible by wounding, including the Agrobacterium pmas promoter (Guevara-Garcia et al. (1993) Plant J. 4(3):495-505) and the Agrobacterium ORF13 promoter (Hansen et al., (1997) Mol. Gen. Genet. 254(3):337-343).

In one aspect, the pollen-ablation construct may be in the form of a plasmid, a vector, a DNA fragment, bacterium, viral vector, or other delivery vehicle. In addition, expression vectors and in vitro culture methods suitable for plant cell or tissue transformation and regeneration of plants are routine and well-known (see, e.g., Gruber et al., “Vectors for Plant Transformation”; at pages 89-119). The cells or plants that contain the construct or multiple constructs may be selected using any suitable marker or technology that allows for its identification or the tracking of the transgenic polynucleotide of interest. One could use any number of techniques known to one of skill in the art to track and breed for the constructs containing one or more transgenic polynucleotide of interest. For example, progeny tests, PCR, molecular markers, or ELISA could be used to trace the transgenic polynucleotides of interest. For example, quantitative PCR could be used to determine which progeny contain which construct and in what dose, and whether it was homozygous or heterozygous for the transgenic polynucleotide of interest. Any technique or combination of techniques may be used.

In one aspect, a plant cell may be transformed with a pollen-ablation construct linked to at least one transgenic polynucleotide of interest and the transformed plant cell generated into a plant. The construct may be introduced to the plant cell using any suitable method, including, but not limited to bombardment, transformation methods, Agrobacterium, silicon carbide fibers, electroporation, microinjection and the like. The cells that have been transformed can be grown into plants in accordance with conventional ways. See, for example, McCormick et al. (1986) Plant Cell Reports 5:81-84. These plants can then be grown and pollinated and resulting plants having expression of the desired phenotypic characteristic can then be identified. Two or more generations can be grown to ensure that expression of the desired phenotypic characteristic is stably maintained and inherited. In another aspect of the invention, the plant cell may be co-transformed with the pollen-ablation construct and a second recombinant construct that expresses a trait or polynucleotide of interest and/or marker. Plant cells expressing both constructs may be selected using any number of methods and the cells generated into plants.

### Example 1

### Vector Constructs and Tobacco Transformation

A vector carrying a translational fusion of the EcoRI restriction endonuclease and G3GFP gene under the control of the pollen-specific LAT52 promoter was constructed with a R4 Gateway Binary Vector (Nakagawa et al., 2008) by using a site-specific multisite Gateway® cloning strategy. The pollen-specific promoter LAT52 derived from tomato has shown to direct high levels of pollen-specific transgene expression with undetectable levels of expression in all other part tissues in several dicotyledonous plants including tobacco (Twell et al., 1990). A plasmid vector carrying the EcoRI restriction endonuclease gene was kindly provided by Linda Jen-Jacobsen at the University of Pittsburg. The EcoRI gene was cloned into TOPO vector (pcr8-GW-TOPO, Invitrogen, Carlsbad, Calif., USA) without the stop codon to enable a reporter gene fusion construct. The LAT52 promoter was cloned into Multisite Gateway® vectors pENTR P4-P1r (Invitrogen, Carlsbad, Calif., USA). All cloned DNA fragments were confirmed for correct orientation using restriction digests and sequence confirmed at the University of Tennessee molecular biology core facility. Tobacco was transformed with Agrobacterium tumefaciens strain EHA105 using an existing tobacco transformation protocol (Horsch et al., 1985). Regenerated events were grown in the greenhouse and T₁ seeds were collected from self-fertilized events. Collected T₁ seeds were surface-sterilized and screened on the medium containing hygromycin 50 mg 1⁻¹.

### Polymerase Chain Reaction (PCR)

Positive T₁ seedlings for hygromycin selection were transplanted in soil and grown in the greenhouse. Two individual plants were grown per each independent event. Genomic DNA was extracted from leaf tissue samples using an existing protocol (Stewart & Via, 1993). The EcoRI gene was amplified with a set of primers (Forward: 5′-ATGTCTAATAAAAAACAGTCAAATA-3′, SEQ ID NO: 1; Reverse: 5′-CTTCTTAGATGTAAGCTGTTC-3′, SEQ ID NO: 2) to confirm transgenicity of selected individuals. The reactions were repeated through 40 cycles of 94° C. for 1 min, 50° C. for 30 s, and 72° C. for 1 min.

### Microscopic Analysis

Pollen samples from each EcoRI T₁ event were visualized to estimate the frequency of GFP positive pollen. Pollen grains from two plants of each EcoRI T₁ event were collected for visual confirmation under an epifluorescence microscopy. Pollen was directly collected from flowers by tapping into a 1.5 ml microfuge tube and was then suspended in sterile water. The microfuge tubes were immediately shaken in a mixer (Eppendorf 5432 mixer) for 10 min to minimize clumping of pollen. The pollen suspension was taken from the tube and placed on a slide glass and covered with a glass cover slip. Pollen screening was performed under an epifluorescence (FITC filtered) microscopy (Olympus BX51 model) with blue light excitation at 200× magnification. QCapture software (Qimaging, Surrey, BC, Canada) was used to acquire pollen images.

### Pollen Viability Analysis

Pollen viability was analyzed by potassium iodide-iodine (IKI) stain method (Mulugeta et al., 1994). One gram of potassium acetate and 0.5 g of iodide were dissolved in sterile H₂O to make IKI staining solution. Fresh pollen grains were collected as previously described. IKI staining solution was added into a 1.5 ml tube containing collected pollen grains. After 30 minutes, 50 μl of pollen grains were sampled on a slide glass and covered with a glass cover slip. Pollen viability was determined under a microscopy (Olympus BX51 model) with white light at 100× magnification.

### Flow Cytometry (FCM)-Based Transgenic Pollen Screen

All plant types including EcoRI T₁ events, non-transgenic Xanthi, and GFP control (CinH_Drec event in Moon et al., 2001b) were grown in the greenhouse. Two individual plants per each event were grown with 2 m distance from other plant types to prevent potential cross-contamination. Pollen collections were conducted for 10 days during flowering period. Collected pollen samples were immediately frozen in liquid nitrogen and stored at −80° C. At the time of assay, 1 ml of sterile water was added into each tube containing pollen grains and the tubes were shaken in a mixer (Eppendorf 5432 mixer) for 10 min. Pollen suspensions were filtered with a 132 μm pore nylon mesh (Sefar Nitex 03-132/43, Sefar filtration Inc., Depew, N.Y., USA) to remove non-pollen debris such as anthers and clumps of pollen that may clog the fluidic system of the flow cytometer. Filtered pollen suspension was transferred into 5 ml polystyrene round bottom tubes (BD Falcon, San Jose, Calif., USA) for FCM analysis. FCM-based transgenic pollen analysis was performed using an existing method described in Moon et al. (2011b). Data were obtained by counting 3,000 pollen grains with 3 technical replicates. Acquired data were analyzed using DiVa software (BD Biosciences, San Jose, Calif., USA) and Cyflogic™ software (CyFlo Ltd, Finland).

Test-Cross with Male Sterile Tobacco

Male sterile (MS) tobacco cv. TN90 seeds were planted as pollen recipient individuals for test-cross. Two individual plants per each EcoRI event were selected on the medium containing hygromycin 50 mg 1⁻¹ and transplanted in soils. Non-transgenic Xanthi and GFP control tobacco were also planted. When the EcoRI events and MS-TN90 were flowering, nine PCR-confirmed EcoRI events were crossed with male-sterile tobacco (cv. TN-90). Non-transgenic Xanthi and GFP control were also crossed with male-sterile tobacco. Manual crosses between the EcoRI events and MS-TN90 were performed to produce seeds. When seed pods on MS-TN90 were mature, seeds were separately harvested from respective MS-TN90 and cleaned. Cleaned seeds were surface-sterilized with 10% bleach and 70% EtOH. Seeds were plated on the medium containing hygromycin 50 mg 1⁻¹. Twenty-eight days after plating the seeds, numbers of total germinated, hygromycin positive and negative seedlings were recorded.

### Results

### Transgenic Tobacco Generation and Phenotype

A transformation vector with a translational fusion of the EcoRI restriction endonuclease and G3GFP gene under the control of the pollen-specific LAT52 promoter was constructed by using a site-specific multisite Gateway® cloning strategyNine independent transgenic EcoRI events were generated via Agrobacterium-mediated transformation method. Transgenicity of T₀ and selected T₁ events were confirmed by PCR. Selected transgenic seedlings on the medium containing hygromycin 50 mg 1⁻¹ were transferred to the greenhouse and grown in soil. Non-transgenic tobacco Xanthi and GFP control plants were grown along with the transgenic events. Throughout the life cycles of the plants, no phenotypic differences between non-transgenic Xanthi and transgenic EcoRI events were observed. Transgenic EcoRI events had normal flowers including petals, sepals, stamens, and carpels. All T₀ EcoRI events produced their T₁ progeny seeds through self-fertilization.

### Pollen Viability

Pollen viability was tested by IKI staining method. Freshly collected pollen grains were stained and screened under an epifluorescence microscopy with 100× magnification. The pollen viability of transgenic EcoRI events was not significantly different from non-transgenic Xanthi and GFP control, except EcoRI-2 event. Only the EcoRI-2 event had statistically different pollen viability compared to non-transgenic Xanthi.

### Microscopic and FCM Analyses

Visual confirmation of transgenic pollen ablation and/or infertility was performed by microscopic analysis. Based on the microscopic pollen images, EcoRI events had significantly less or no GFP positive pollen grains compared to the GFP control. To accurately analyze large numbers of pollen grains, FCM-based transgenic pollen screening method was used. All EcoRI events had less that 0.5% of GFP positive pollen grains, while GFP control had 64% of GFP positive pollen. Since GFP control plants were mixed population of hemizygous and homozygous for GFP transgene, it was expected to have over 50% of GFP positive pollen grains. Non-transgenic Xanthi contained 0% of GFP positives as expected. Three EcoRI events, EcoRI-5, 7, and 8, had 0% of GFP positives. This result suggests that these events could have perfect selective male sterility.

### Test-Cross

Theoretically, if transgenic pollen were successfully ablated or infertile, there would be no homozygous lines produced in T₁ generation. Positive T₁ seedlings of each EcoRI event were selected on hygromycin selection medium and grown in soils. No seeds were produced from MS-TN90 plants that have not been crossed with other tobacco. Except the EcoRI-4 and 9 events, all other EcoRI events had 100% hygromycin negative seeds from the test-crosses. All test-crossed EcoRI seeds, except the EcoRI-4 and 9 events, were dead on the selective medium. This suggests that pollen from these EcoRI events excluding the EcoRI-4 and 9 events did not carry transgenes. This result indicates that transgenic pollen was successfully ablated and/or being infertile by overexpression of the EcoRI restriction endonuclease in pollen. This approach could be used as an effective biocontainment strategy.

### Discussion

Transgenic pollen was successfully ablated and/or rendered infertile by overexpression of the EcoRI restriction endonuclease in pollen. The type II restriction endonuclease EcoRI was used to destruct nuclear genomic DNA in pollen. Pollen specific LAT52 promoter drove a translational fusion of the EcoRI and GFP gene. Based on the FCM-based transgenic pollen screening result, three events, EcoRI-5, 7, and 8 had 100% efficiency on selective male sterility. However, further confirmation with the test-cross showed that most of the EcoRI events had higher efficiency on selective male sterility compared to the ones from the FCM-based pollen analysis. Seven independent EcoRI events had 100% efficiency on selective male sterility. This suggests that this selective male sterility approach could result in a perfect prevention of transgenic pollen-mediated transgene escape. This approach could be used as a highly efficient biocontainment strategy. Complete genic or cytoplasmic male sterility could cause environmentally negative impacts on pollen-feeding insects. This selective male sterility approach could be considered as the most environmentally friendly strategy unlike other complete male sterility approaches since it could serve as a food source for pollen-feeding insects by producing plenty of non-transgenic pollen. There might be concerns regarding potential negative effects of restriction endonuclease on insect pollinators. If transgenic pollen were completely ablated, insect pollinators would not be exposed to restriction endonulcease. In case transgenic pollen rendered infertility while being formed, there might be an environmental safety issue. However, restriction endonulcease activity is not only related to the presence or absence of the respective restriction sites in chromosome (Tartarotti et al., 2000). The restriction endonuclease EcoRII did not attack satellite heterochromatin in cytological preparations of mouse chromosomes, even though it showed a capability of cleavage with extensively purified mouse satellite DNA (Southern, 1975). Chromatin structures of different species can be an important factor that affects the activity of the restriction endonucleases in eukaryotic chromosomes (Gosalvez et al., 1989; Petitpierre et al., 1996).

Cytotoxic genes were used in most male sterility studies to prevent pollen formation. Using these cytotoxic genes might be a concern of potential toxicity to non-targeted organisms or cells. This potential toxicity would not be a problem if the EcoRI restriction endonuclease that is not toxic to cells were used for male sterility.

One of the most environmentally friendly strategies, transgene excision in pollen, is being debated on its effectiveness and sustainability because it leaves some parts of T-DNA after transgene excision occurs. In case of site-specific recombinase-mediated transgene excisions, several parts of T-DNA including left and right borders and a recognition site of site-specific recombianse that have been integrated into plant genome through genetic engineering would remain in pollen genome. Transgene excision in pollen is arguable because it technically does not result in completely transgene-free pollen. However, this selective male sterility would not produce any pollen that carries a small portion of T-DNA even if those are not functional. This approach could be an ideal biocontainment strategy for environmental and regulatory concerns on transgenic crops.

Non-transgenic Xanthi contained 0% GFP positive pollen from the FCM-based pollen analysis unlike the previously reported studies (Moon et al., 2011a; 2011b). It suggested that GFP positive pollen contamination in non-transgenic pollen reported in the previous studies could come from cross-pollination between transgenic events and non-transgenic Xanthi in the greenhouse.

Based on the result of the test-cross, seven independent events had 100% efficiency on selective male sterility. While only three events demonstrated 100% efficiency from the FCM-based transgenic pollen screening method. Since FCM-based transgenic pollen screening method relies on GFP positive pollen, it is possible that previously synthesized GFP was exhibited in pollen, so it was considered as a GFP positive. However, it became infertile due to the destruction of pollen genome afterwards. In this case, FCM-based transgenic pollen screening method could possibly underestimate the efficiency of transgenic pollen ablation and/or infertility.

Pollen recipient male-sterile (MS) tobacco was an effective tool for an initial screening the efficacy of events in the greenhouse. To acquire accurate progeny data for biocontainment strategies including transgene excision approach, emasculated tobacco plants might be required as pollen recipient plants (Moon et al., 2011a). No seeds were produced from male-sterile tobacco plants that have not been crossed with other tobacco. It assures that all produced seeds were resulted from a cross between respective transgenic EcoRI events and MS tobacco.

If transgenic pollen grains are not fertile owing to the destruction of pollen genome, EcoRI events should have higher percentage of non-viable pollen than non-transgenic Xanthi. On the other hand, if there is no significant difference in pollen viability between EcoRI events and non-transgenic Xanthi, pollen would most likely being ablated. Once transgenic pollen is ablated, collected pollen from EcoRI event that is assumed to be non-transgenic would not be apparently different from that of non-transgenic Xanthi. The result of pollen viability test indirectly indicates that pollen ablation might occur, since most of the events had similar percentage of non-viable pollen with non-transgenic Xanthi. However, direct evidence such as pollen count is required to more clearly address this question. From the results, it is clear that pollen from transgenic plants overexpressing the EcoRI restriction endonuclease in pollen would not carry transgenes. These results indicate that overexpression of the EcoRI restriction endonuclease could cause transgenic pollen ablation and/or infertility. This selective male sterility could be used as an efficient and reliable biocontainment strategy to eliminate pollen-mediated transgene escape and introgression.

### Example 2

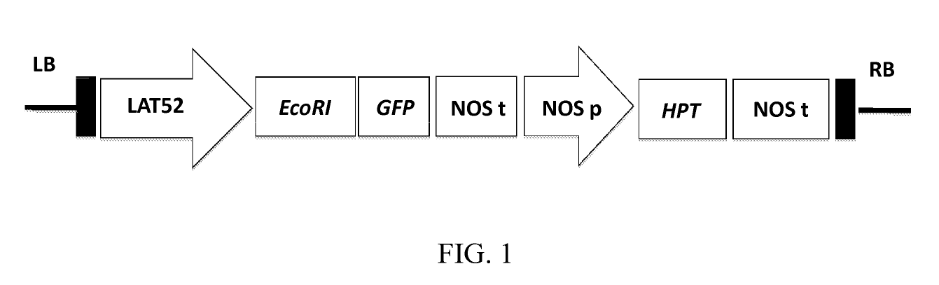

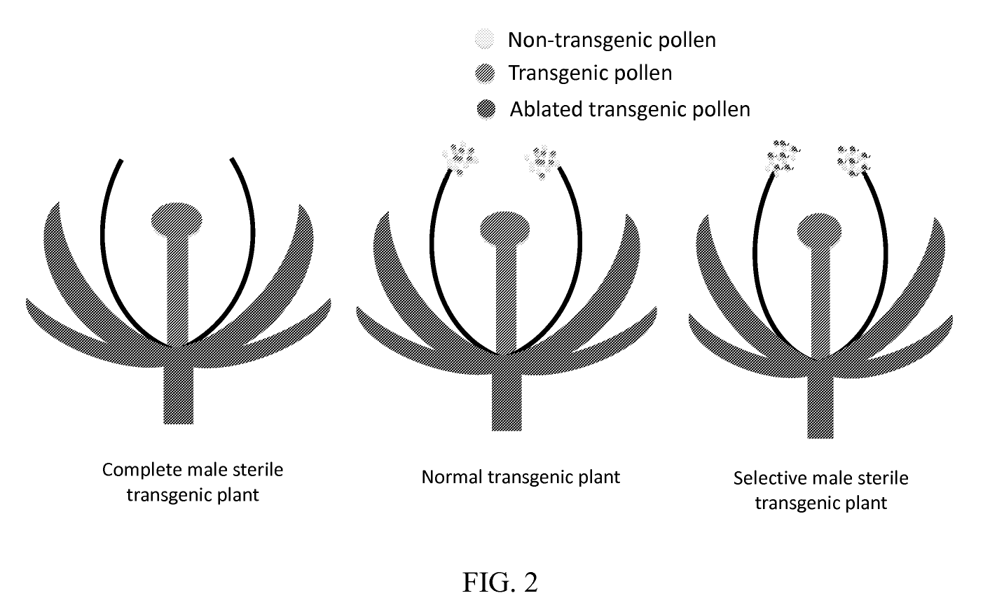

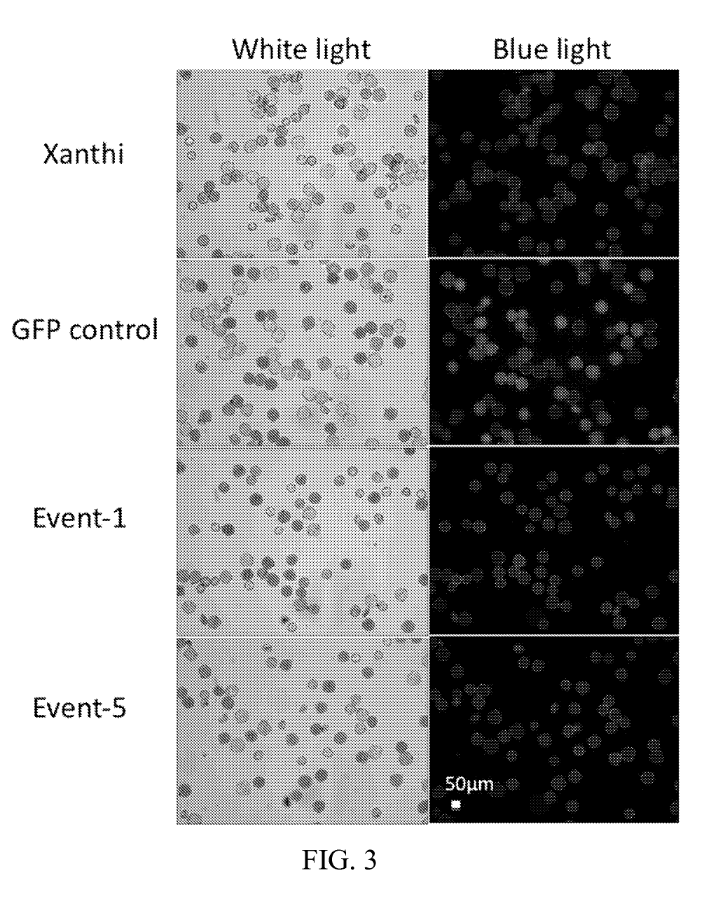

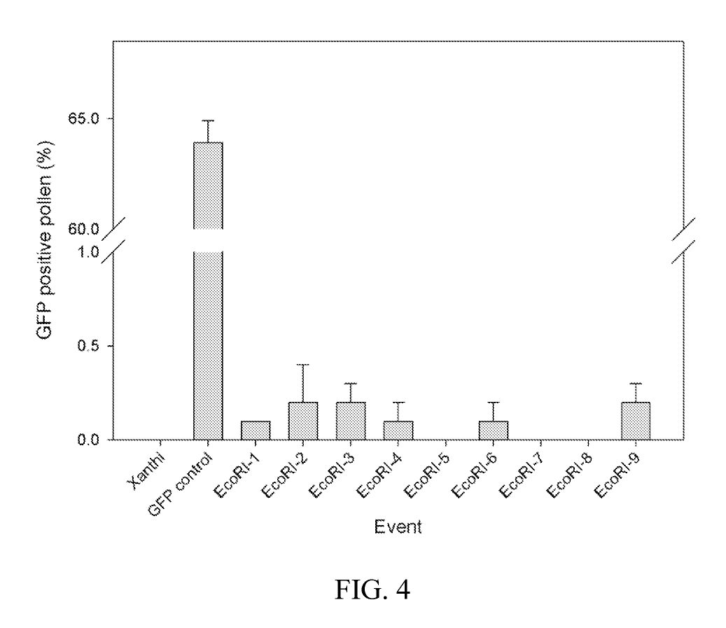

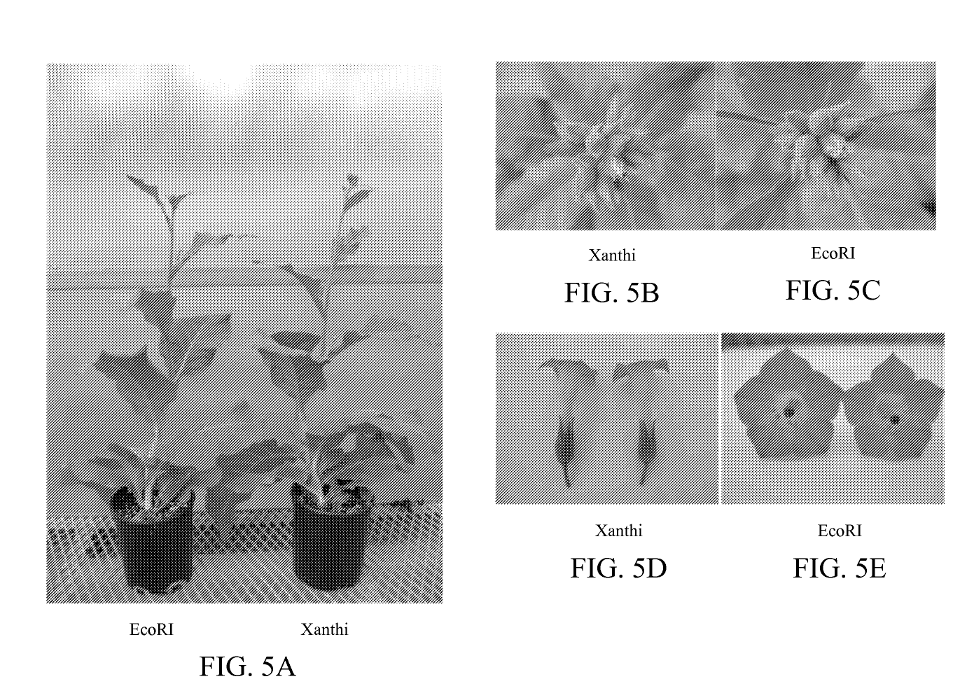

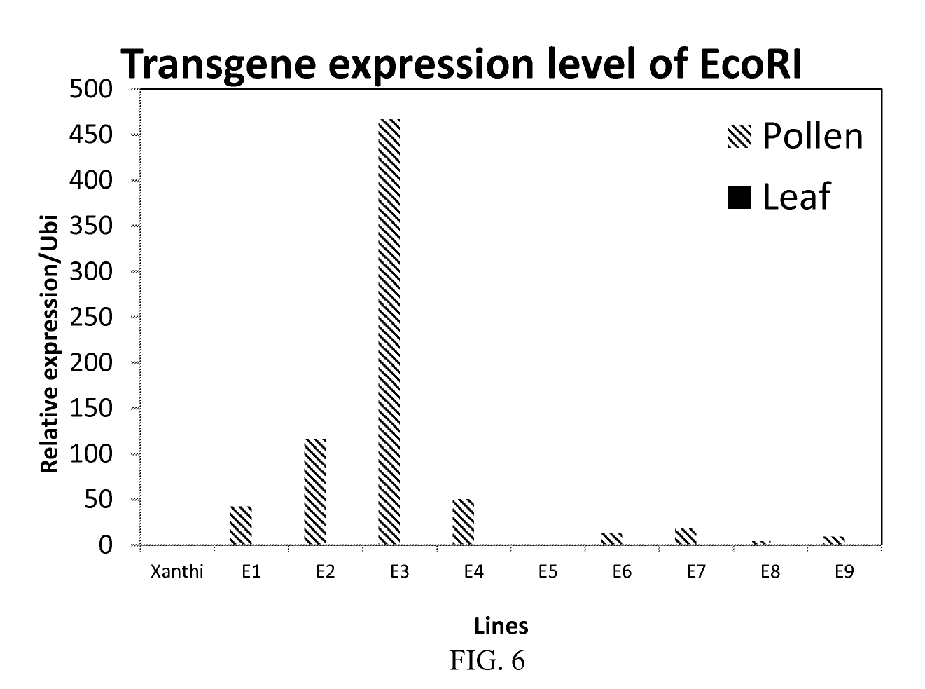

Type II restriction endonucleases, such as EcoRI are very well characterized and have long-served as workhorses for molecular biology. We placed an EcoRI-GFP fusion gene under the control of a pollen-specific promoter, which was stably integrated in transgenic tobacco. The resultant plants were self-fertilized and a flow cytometry method detected very few GFP-positive pollen grains, yet some events had decreased pollen viability. A test-cross to a male-sterile tobacco mutant (used as the female parent) was made and seeds were collected from the male-sterile parent and germinated on selective media and PCR-confirmed. Several of the transgenic lines displayed 100% efficacy for the male-sterile trait (of ˜40,000 seeds plated). Pollen-specific EcoRI expression appears to be an effective, and potentially universal pollen ablation tool that could be used in any plant species for transgene bioconfinement. In addition to its efficacy, there is no indication of any off-target effects to the plant, since plants appeared morphologically normal. Field experiments are currently being performed with test-crosses in the same tobacco system and EcoRI with other pollen-specific promoter constructs is being engineered into other plant species. FIGS. 1-6 illustrate that restriction enzyme mediated pollen ablation appears to be an effective species-independent mal sterility and bioconfinement tool. As demonstrated by the Figures, GFP fluorescence was detected in the pollen of EcoRI transgenics but at a significantly lower rate than GFP positive controls and biocontainment efficiencies were very high in the test-cross (Table 1). The Figures also illustrate that it is evident that EcoRI expression in pollen is a viable option effective biocontainment and hybridization tool. Furthermore, there were no phenotype difference found between EcoRI expressing plants and nontransgenics and EcoRI transcripts were detected in pollen, but not in other assayed tissues.

TABLE 1
Test-cross results. EcoRI plants were hand-crossed with male-sterile tobacco. The
male-sterile plants were used as the maternal parent. Progeny were screened on media
containing hygromycin and PCR confirmed for the EcoRI gene.

| | | Seeds | Seeds | Putative | PCR Confirmed | Biocontainment |
|---|---|---|---|---|---|---|
| Lines | Medium | plated | germinated | Hyg Resistant | for EcoRI | Efficiency |
| Xanthi | MSO + Hyg | 38800 | 36860 | 0 | 0 | N/A |
| El | MSO + Hyg | 40000 | 38000 | 1 | 0 | 100% |
| E2 | MSO + Hyg | 29800 | 28310 | 6 | 0 | 100% |
| E3 | MSO + Hyg | 40000 | 38000 | 2 | 0 | 100% |
| E4 | MSO + Hyg | 40000 | 38000 | 336 | 254 | 99.332% |
| E5 | MSO + Hyg | 41000 | 38950 | 6 | 2 | 99.995% |
| E6 | MSO + Hyg | 42000 | 39900 | 1 | 0 | 100% |
| E7 | MSO + Hyg | 40000 | 38000 | 9 | 3 | 99.982% |
| E8 | MSO + Hyg | 23600 | 22420 | 74 | 37 | 99.835% |
| E9 | MSO + Hyg | 16000 | 15200 | 24 | 19 | 99.875% |

What is claimed is:

1. A recombinant pollen-ablation construct comprising a pollen-specific promoter operably linked to a polynucleotide encoding a restriction enzyme, wherein said pollen-ablation construct renders the pollen unable to fertilize a sexually compatible ovule.

2. The recombinant pollen-ablation construct according to claim 1, wherein the pollen-ablation construct is linked to at least one transgenic polynucleotide of interest.

3. The recombinant pollen-ablation construct according to claim 2, wherein the transgenic polynucleotide of interest is selected from the group consisting of a polynucleotide impacting insecticide resistance, disease resistance, herbicide resistance, drought tolerance, cold tolerance, nitrogen utilization, nutrition content, cellulose content, male sterility, female sterility, abiotic stress resistance, antibiotic resistance, site specific DNA integration, and a marker.

4. The recombinant pollen-ablation construct according to claim 3, wherein the transgenic polynucleotide of interest is a polynucleotide confers herbicide resistance to glyphosphate, glyfosinate, a sulfonylurea herbicide, an imidazoline herbicide, a hyrdoxyphenylpyruvate dioxygenase inhibitor, or a protoporphyrinogen oxidase inhibitor.

5. The recombinant pollen-ablation construct according to claim 2, wherein the transgenic polynucleotide of interest is a polynucleotide encodes a Bacillus thuringienesis toxin.

6. The recombinant pollen-ablation construct according to claim 1, wherein the construct further comprises a marker.

7. The recombinant pollen-ablation construct according to claim 2, wherein the construct comprises a second promoter that drives the expression of the transgenic polynucleotide of interest.

8. The recombinant pollen-ablation construct according to claim 7, wherein the second promoter is selected a constitutive or inducible promoter.

9. A plant comprising a recombinant pollen-ablation construct according to claim 1.

10. The plant according to claim 9, wherein said plant is an angiosperm or gymnosperm.

11. The plant according to claim 9, wherein the plant is corn, maize, canola, tobacco, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, switchgrass, rapeseed, clover, tobacco, turfgrass, carrot, cotton, alfalfa, rice, potato, eggplant, cucumber or Arabidopsis thaliana.

12. Seeds obtained from a plant according to claim 9.

13. An isolated cell comprising a recombinant pollen-ablation construct according to claim 1.

14. Pollen or ovules of the plant according to claim 9.

15. A vector comprising a recombinant pollen-ablation construct according to claim 1.

16. A method for producing a plant that will not produce functional transgenic pollen comprising: transforming a plant cell with a recombinant pollen-ablation construct according to claim 1 and regenerating a plant from the transformed plant cell.

17. The method according to claim 16, further comprising propagating the generated plant to produce progeny.

18. A transgenic plant produced by the method according to claim 17.

19. Seeds obtained from the plant produced by the method according to claim 17.

20. The recombinant pollen-ablation construct according to claim 1, wherein said restriction enzyme is a type I, type II or type III restriction endonuclease.

21. The recombinant pollen-ablation construct according to claim 20, wherein said restriction enzyme is AarI; BanII; BseGI; BspPI; CfrI; EcoNI; Hsp92II; NlaIV; RsaI; TaiI; AasI; BbsI; BseJI; BspTI; ClaI; EcoO109I; I-PpoI; NmuCI; RsrII; TaqaI; AatII; BbuI; BseLI; BsrBI; CpoI; EcoRI; KasI; NotI; SacI; TaqI; Acc65I; BbvCI; BseMI; BsrDI; Csp45I; EcoRV; Kpn2I; NruI; SacII; TasI; AccB7I; BbvI; BseMII; BsrFI; Csp6I; EheI; KpnI; NsbI; SalI; TatI; AccI; BceAI; BseNI; BsrGI; CspI; Esp3I; KspAI; NsiI; SapI; TauI; AccIII; BcgI; BseRI; BsrI; DdeI; FauI; LweI; NspI; SatI; TfiI; AciI; BciVI; BseSI; BsrSI; DpnI; Fnu4HI; MbiI; OliI; Sau3AI; TliI; AclI; BclI; BseXI; BssHII; DpnII; FokI; MboI; PacI; Sau96I; TrulI; AdeI; BcnI; BseYI; BssKI; DraI; FseI; MboII; PaeI; SbfI; Tru9I; AfeI; BcuI; BsgI; BssSI; DraIII; FspAI; MfeI; PaeR7I; ScaI; TseI; AflII; BfaI; Bsh1236I; Bst1107I; DrdI; FspI; MlsI; PagI; SchI; Tsp45I; AflIII; BfiI; Bsh1285I; Bst98I; EaeI; GsuI; MluI; PauI; ScrFI; Tsp509I; AgeI; BfmI; BshNI; BstAPI; EagI; HaeII; MlyI; PciI; SdaI; TspRI; AhdI; BfrBI; BshTI; BstBI; Eam1104I; HaeIII; MmeI; PdiI; SduI; Tth111I; AleI; BfuAI; BsiEI; BstEII; Eam1105I; HgaI; MnlI; PdmI; SexAI; TurboNael; AloI; BfuCI; BsiHKAI; BstF5I; EarI; HhaI; Mph1103I; Pfl231I; SfaNI; TurboNarI; AluI; BfuI; BsiWI; BstNI; EciI; HinlI; MscI; PflFI; SfcI; Van91I; Alw21I; BglI; BslI; BstOI; Ecl136II; Hin4I; MseI; PflMI; SfiI; VspI; Alw26I; BglII; BsmAI; BstUI; EclHKI; Hin6I; MslI; PfoI; SfoI; XagI; Alw44I; BlpI; BsmBI; BstXI; Eco105I; HincII; MspAlI; PleI; Sgfl; XapI; AlwI; Bme1390I; BsmFI; BstYI; Eco130I; HindIII; MspI; PmeI; SgrAI; XbaI; AlwNI; BoxI; BsmI; BstZI; Eco147I; HinfI; MssI; PmlI; SinI; XceI; ApaI; BpiI; BsoBI; Bsu15I; Eco24I; HinPlI; MunI; PpiI; SmaI; XcmI; ApaLI; BplI; Bsp119I; Bsu36I; Eco31I; HpaI; Mva1269I; PpuMI; SmiI; XhoI; ApoI; Bpu10I; Bsp120I; BsuRI; Eco32I; HpaII; MvaI; PshAI; SmlI; XhoII; AscI; Bpu1102I; Bsp1286I; BtgI; Eco47I; HphI; MwoI; PsiI; SmuI; XmaI; AseI; BsaAI; Bsp1407I; BtsI; Eco47III; Hpy188I; NaeI; Psp1406I; SnaBI; XmaJI; AsiSI; BsaBI; Bsp143I; BveI; Eco52I; Hpy188III; NarI; Psp5II; SpeI; XmiI; AvaI; BsaHI; Bsp143II; Cac8I; Eco57I; Hpy8I; NciI; PspGI; SphI; XmnI; AvaII; BsaI; Bsp68I; CaiI; Eco57MI; Hpy99I; NcoI; PspOMI; SspI; AvrII; BsaJI; BspDI; CfoI; Eco72I; HpyCH4III; NdeI; PstI; StuI; BaeI; BsaMI; BspEI; Cfr10I; Eco81I; HpyCH41V; NdeII; PsuI; StyD4I; BalI; BsaWI; BspHI; Cfr13I; Eco88I; HpyCH4V; NgoMIV; PsyI; StyI; BamHI; BsaXI; BspLI; Cfr42I; Eco91I; HpyF1VI; NheI; PvuI; SwaI; BanI; BseDI; BspMI; Cfr9I; EcoICRI; Hsp92I; N1aIII; PvuII; or TaaI.

22. The recombinant pollen-ablation construct according to claim 21, wherein said restriction enzyme is AluI; ClaI; Eco47III; HaeIII; KpnI; NdeI; PstI; SacII; SfiI; XhoI; BamHI; DpnI; EcoRI; HindIII; MspI; NheI; RsaI; SalI; SmaI; XmaI; BglII; DpnII; EcoRV; HpaII; NcoI; NotI; SacI; Sau3AI; or XbaI.

23. The recombinant pollen-ablation construct, pollen according to claim 22, wherein said restriction enzyme is EcoRI.
